(19) United States
(12) Patent Application Publication
(10) Pub. No.: US 2015/0100014 A1
(43) Pub. Date: Apr. 9, 2015

(54) FLUID INFUSING APPARATUS AND TRANSPORTING STATE DETERMINATION METHOD

(71) Applicant: SEIKO EPSON CORPORATION, Tokyo (JP)

(72) Inventors: Yukihiro UCHIYAMA, Okaya-shi (JP); Yoshihiko MOMOSE, Shiojiri-shi (JP)

(21) Appl. No.: 14/498,473

(22) Filed: Sep. 26, 2014

(30) Foreign Application Priority Data
Oct. 3, 2013 (JP) 2013-207970

(51) Int. Cl. A61M 5/172; G01M 99/00; G01B 21/16; A61M 5/168

(52) U.S. Cl. CPC A61M 5/172; A61M 5/16886; G01M 99/00; G01B 21/16; A61M 2205/702
USPC 604/67; 73/865.9

(57) ABSTRACT

A fluid infusing apparatus includes: a flow channel member configured to transport a fluid; a cylindrical portion provided on the flow channel member, a measuring unit configured to measure a displacement of the cylindrical portion; a determining unit configured to determine a condition of transport of the fluid on the basis of the displacement of the cylindrical portion.

### BACKGROUND

1. Technical Field

The present invention relates to a fluid infusing apparatus configured to infuse fluid and a transporting state determination method.

2. Related Art

An insulin pump configured to inject insulin into a biological body is now put into practical use. A fluid infusing apparatus such as the insulin pump is fixed to the biological body such as human body, and injects a fluid into the biological body such as the human body regularly according to a preset program.

JP-A-2011-174394 discloses a technology to determine a condition of transport of the fluid on the basis of a measurement of a change in capacitance between a pair of electrodes provided with a tube which constitutes a flow channel interposed therebetween.

In the technology disclosed in JP-A-2011-174394, a change in capacitance is detected. However, since an amount of change in capacitance is very small, a measuring instrument of a high precision is required. In addition, in the case where a measurement instrument having a general precision is used, time until the amount of change in capacitance reaches a measurable level is required. Therefore, it is desired to allow a condition of transport of the fluid to be determined by other methods.

### SUMMARY

An advantage of some aspects of the invention is to determine a condition of transport of a fluid.

An aspect of the invention provides a fluid infusing apparatus including: a flow channel member configured to transport a fluid; a cylindrical portion provided on the flow channel member; a measuring unit configured to measure a displacement of the cylindrical portion; a determining unit configured to determine a condition of transport of the fluid on the basis of the displacement of the cylindrical portion.

Other characteristics of the invention will be apparent from the specification and attached drawings.

### BRIEF DESCRIPTION OF DRAWINGS

The invention will be described with reference to the accompanying drawings, wherein like numbers reference like elements.

FIG. 1 is a general perspective view of a micro pump.

FIG. 2 is an exploded view of the micro pump.

FIG. 3 is a perspective top view of the micro pump.

FIG. 4 is a cross-sectional view of the micro pump.

FIG. 5 is an internal perspective view of a main body.

FIG. 6 is a perspective view of a back surface of the main body.

FIG. 7 is an exploded perspective view of a cartridge.

FIG. 8 is a perspective view of a back surface of a cartridge base.

FIG. 9 is a perspective view of a back surface of the micro pump.

FIG. 10 is an explanatory drawing of a rotary finger pump.

FIG. 11 is a block diagram of a control unit in the micro pump of a first embodiment.

FIG. 12 is a first cross-sectional view taken along a B-B line in FIG. 3 (first embodiment).

FIG. 13 is a first cross-sectional view taken along a C-C line in FIG. 3 (first embodiment).

FIG. 14 is a second cross-sectional view taken along the B-B line in FIG. 3 (first embodiment).

FIG. 15 is a second cross-sectional view taken along the C-C line in FIG. 3 (first embodiment).

FIG. 16 is a block diagram of a control unit of a micro pump of a second embodiment.

FIG. 17 is a first cross-sectional view taken along the B-B line in FIG. 3 (second embodiment).

FIG. 18 is a first perspective view of a pressure detecting member (second embodiment).

FIG. 19 is a second cross-sectional view taken along the B-B line in FIG. 3 (second embodiment).

FIG. 20 is a second perspective view of the pressure detecting member (second embodiment).

### DETAILED DESCRIPTION

According to the specification and the accompanying drawings, at least the followings become apparent. That is, a fluid infusing apparatus includes: a flow channel member configured to transport a fluid; a cylindrical portion provided on the flow channel member; a measuring unit configured to measure a displacement of the cylindrical portion; a determining unit configured to determine a condition of transport of the fluid on the basis of the displacement of the cylindrical portion.

In this configuration, if a clogging occurs downstream of the flow channel, an internal pressure in the cylindrical portion is increased and the cylindrical portion itself is deformed. Therefore, a displacement of the cylindrical portion is measured, and the condition of transport of the fluid may be determined on the basis of the amount of displacement obtained by measurement. It is noted that the determined condition of transport of the fluid includes not only a concept of the condition at a certain point but also a concept of a change the condition.

In the fluid infusing apparatus, it is preferable that the measuring unit includes at least one of an ultrasonic sensor and a strain gauge.

With this configuration, a displacement of a film-shaped member can be measured indirectly or directly.

In the fluid infusing apparatus, it is preferable that an air layer is provided between a lid portion provided on the cylindrical portion and the fluid.

Air in the air layer is readily compressed in comparison with the fluid, and hence even in the case where an abrupt change is generated in the condition of transport of the fluid, the abrupt change can be alleviated by the air layer. Accordingly, a damage generated from an excessive deformation of the cylindrical portion may be restrained.

In the fluid infusing apparatus, it is preferable that rigidity of the cylindrical portion is lower than rigidity of the flow channel member.

With this configuration, since the rigidity of the cylindrical portion is lower than the rigidity of the flow channel member, when the clogging occurs in a tube connected to downstream side of the flow channel, and hence the internal pressure of the flow channel is increased, the amount of deformation of the cylindrical portion is larger than that of the flow channel member. Therefore, by measuring the amount of displacement of the cylindrical portion, the condition of transport of the fluid can be determined with higher sensitivity.

In the fluid infusing apparatus, it is preferable that a thickness of the cylindrical portion is lower than a thickness of the flow channel member.

With this configuration, the rigidity of the cylindrical portion can be reduced to be lower than the rigidity of the flow channel member. Accordingly, the condition of transport of the fluid can be determined with higher sensitivity.

In the fluid infusing apparatus, it is preferable that rigidity of the tube to be connected to the flow channel member is higher than at least the rigidity of the cylindrical portion.

With this configuration, since the rigidity of the tube to be connected to the flow channel member is higher than the rigidity of the cylindrical portion, the cylindrical portion is deformed more than the tube in the case where a clogging occurs in the tube connected to downstream side of the flow channel member. Therefore, by measuring the amount of displacement of the cylindrical portion, the condition of transport of the fluid can be determined with higher sensitivity.

In the fluid infusing apparatus, it is preferable that the cylindrical portion extends from the flow channel member in a direction intersecting the flow channel of the flow channel member.

With this configuration, the cylindrical portion capable of readily having the air layer can be provided.

According to the specification and the accompanying drawings, at least the followings become apparent as well. That is, a transporting state determination method for a fluid in a fluid infusing apparatus including: a flow channel member configured to transport the fluid; and a cylindrical portion provided on the flow channel member, includes: measuring a displacement of the cylindrical portion; and determining a condition of transport of the fluid on the basis of the measured displacement of the cylindrical portion.

With this configuration, if a clogging occurs downstream of the flow channel, an internal pressure in the cylindrical portion is increased and the cylindrical portion itself is deformed. Therefore, a displacement of the cylindrical portion is measured, and the condition of transport of the fluid may be determined on the basis of the amount of displacement obtained by measurement.

### First Embodiment

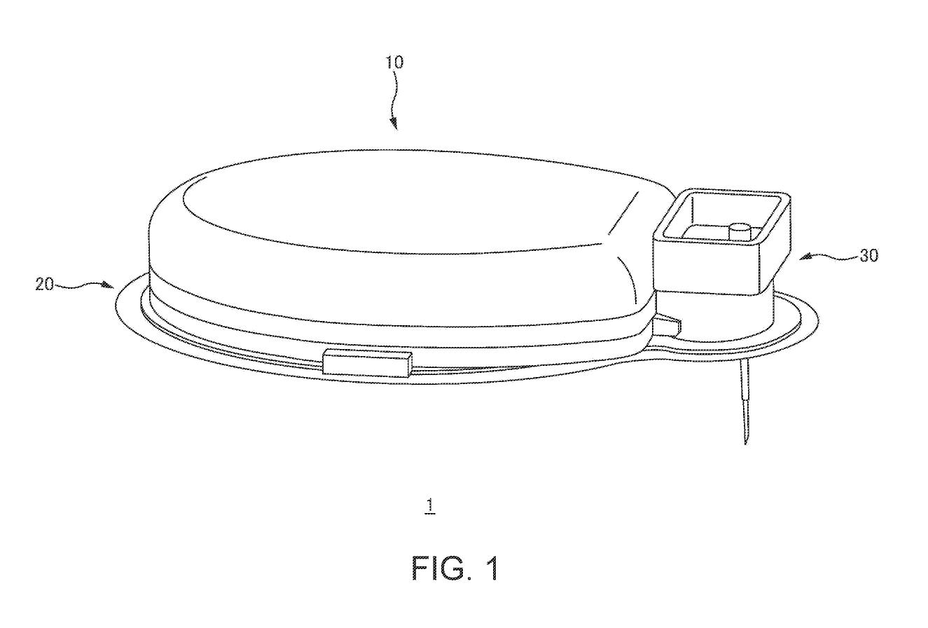

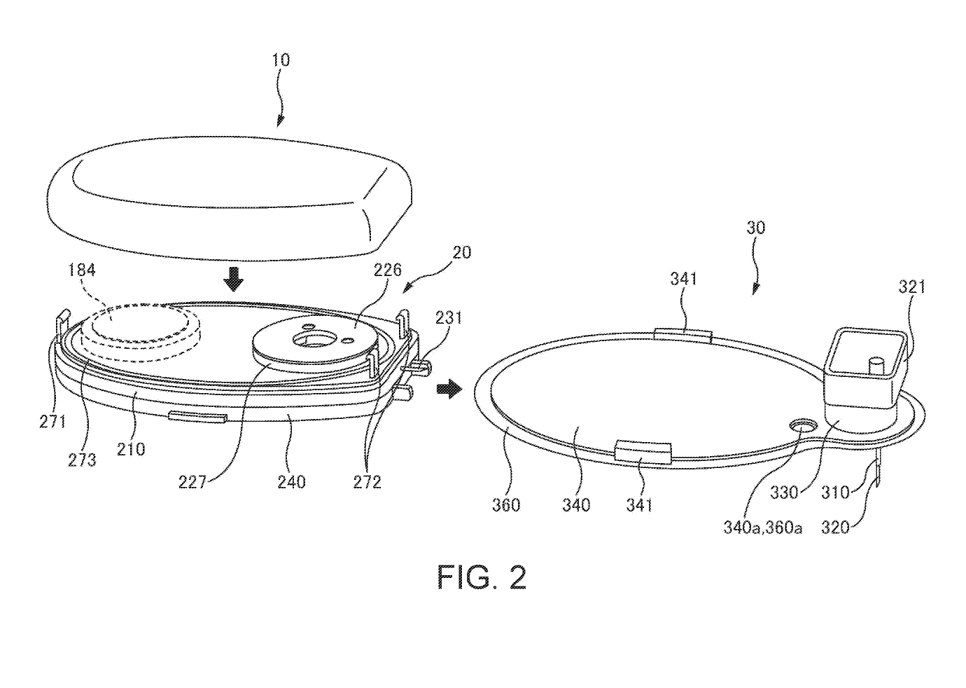

FIG. 1 is a general perspective view of a micro pump 1. FIG. 2 is an exploded view of the micro pump 1. The micro pump 1 includes a main body 10, a cartridge 20, and a patch 30. These three members may be separated as illustrated in FIG. 2. However, when in use, these members are assembled integrally as illustrated in FIG. 1. The micro pump 1 is adhered to a biological body, and is preferably used for regular infusion of insulin.

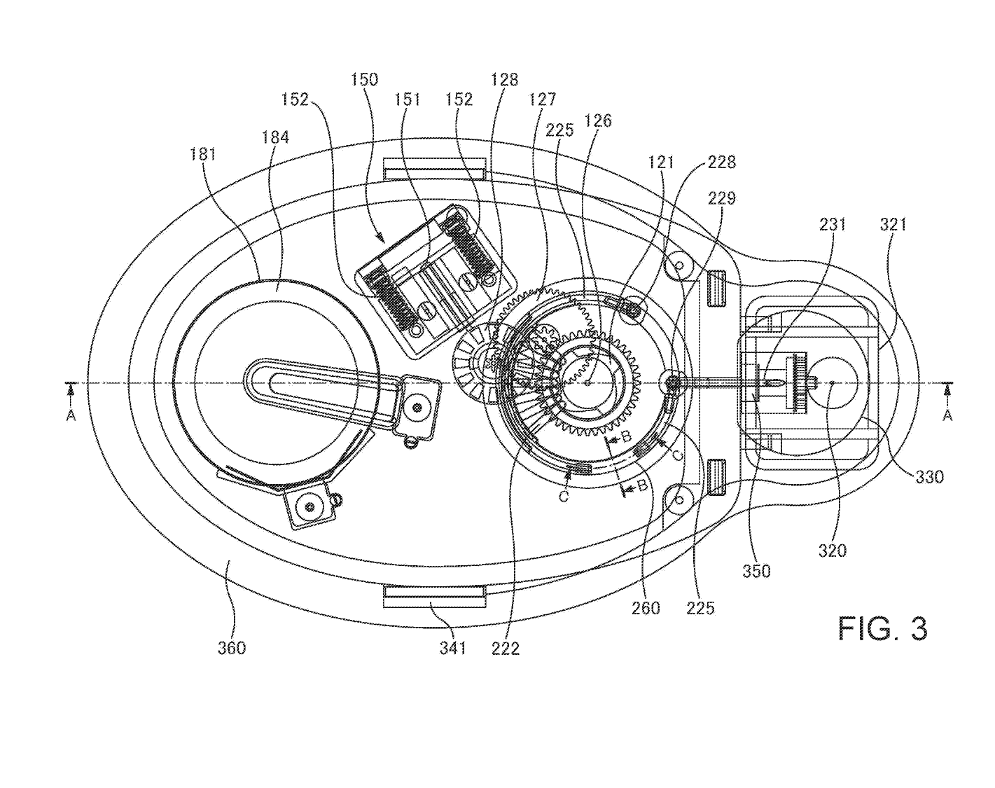

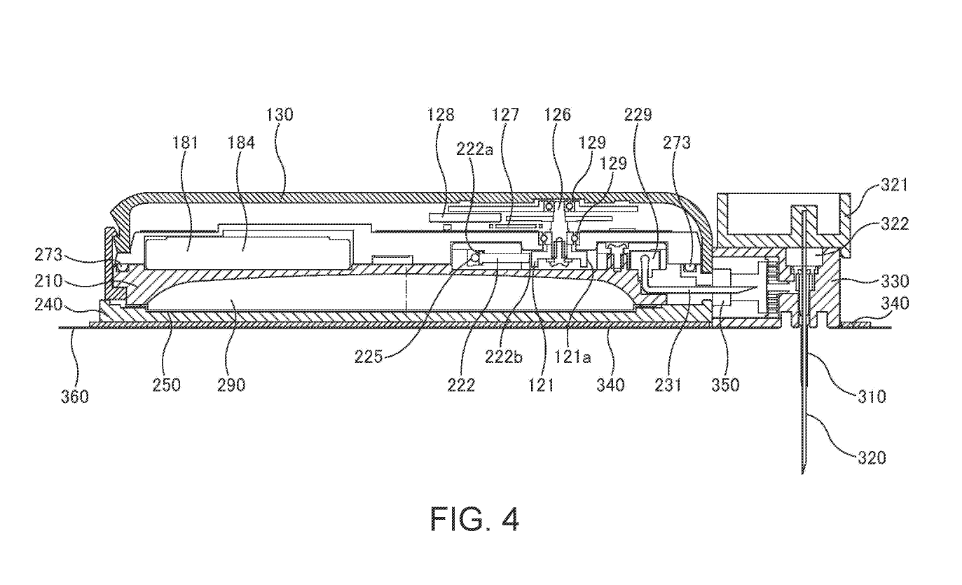

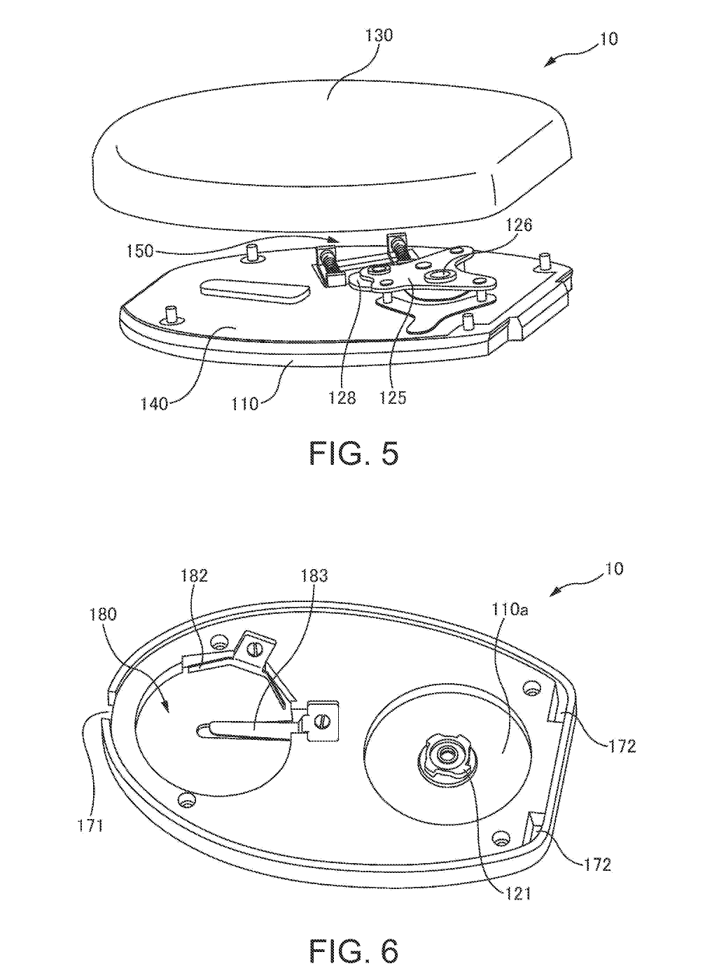

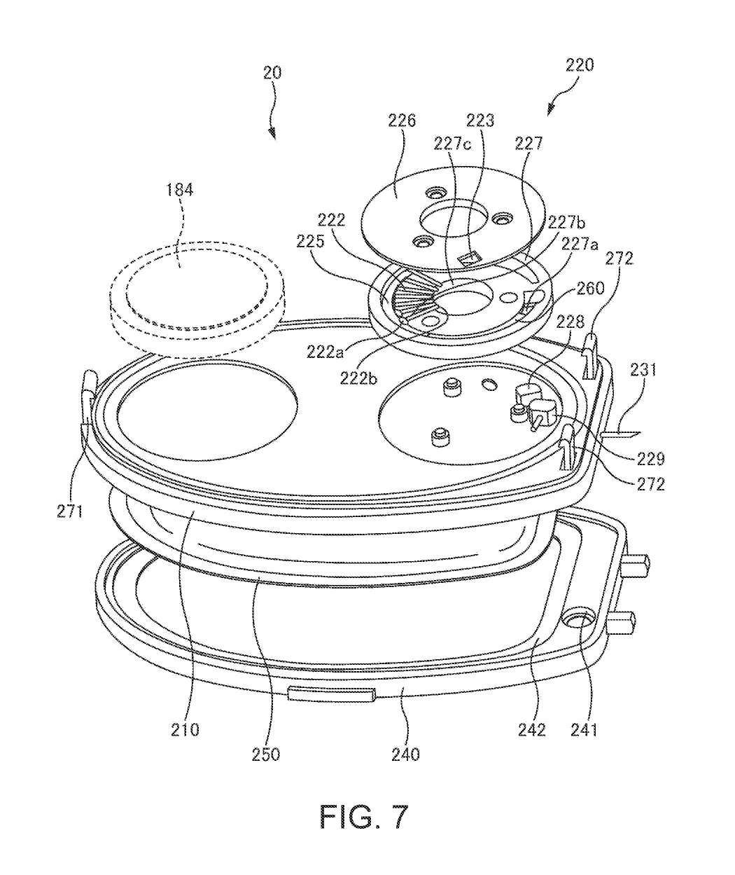

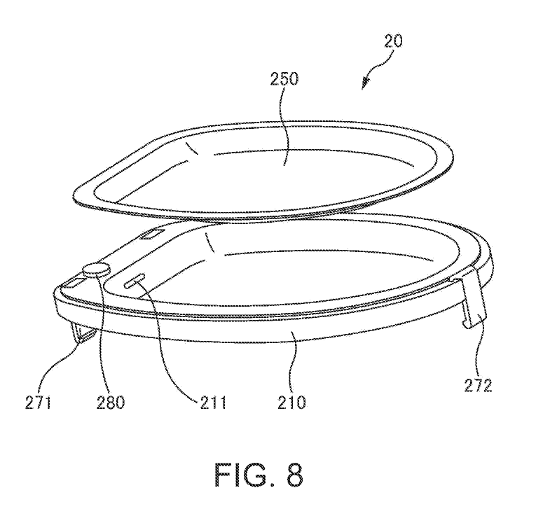

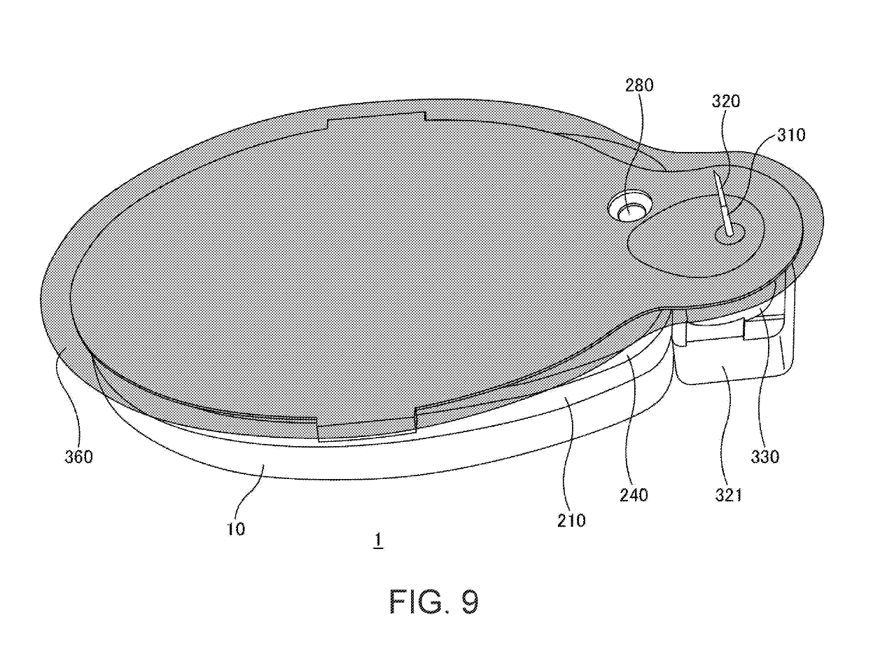

FIG. 3 is a perspective top view of the micro pump 1. FIG. 4 is a cross-sectional view of the micro pump 1. In other words, FIG. 3 and FIG. 4 are drawings illustrating an assembled state of the main body 10, the cartridge 20, and the patch 30. FIG. 5 is an internal perspective view of the main body 10. FIG. 6 is a perspective view of a back surface of the main body 10. FIG. 6 is a drawing illustrating a back surface of FIG. 5 described above. FIG. 7 is an exploded perspective view of the cartridge 20. FIG. 8 is a perspective view of a back surface of a cartridge base 210. FIG. 9 is a perspective view of a back surface of the micro pump 1.

With reference to FIG. 1 to FIG. 9, respective members of the micro pump 1 will be described below. First of all, the respective members of the main body 10 will be described.

As illustrated in FIG. 1, the micro pump 1 includes the main body 10, the cartridge 20, and the patch 30 as principal components.

As illustrated in FIG. 5, the main body 10 includes a main body base 110, respective components provided on the main body base 110, and a main body case 130. The respective components on the main body base 110 is covered with the main body case 130 and protected thereby.

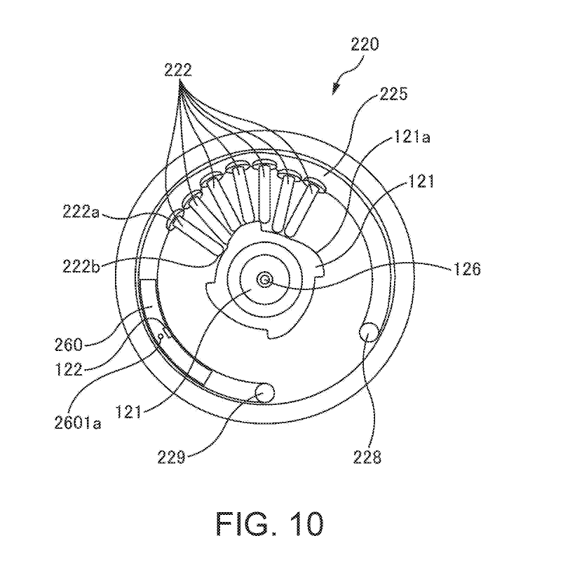

The main body 10 includes a circuit substrate 140 provided on the main body base 110. The circuit substrate 140 is an electronic substrate for controlling a piezoelectric motor 150 or the like according to a program or the like, and includes a control unit 141. The main body includes the piezoelectric motor 150. The piezoelectric motor 150 is a motor for providing a cam 121, which will be described later, with a rotational drive force (FIG. 10).

As illustrated in FIG. 3, the piezoelectric motor 150 includes a plate-shaped member 151 and a pair of springs 152. The springs 152 bias the plate-shaped member 151 toward a rotor gear 128 by a resilient force thereof. The plate-shaped member 151 is biased toward the rotor gear 128 as described above, and a distal end portion thereof comes into contact with a peripheral surface of the rotor gear 128.

The plate-shaped member 151 is a member formed into a layer. The plate-shaped member 151 includes a piezoelectric layer and two electrodes, and is changed in shape by a change of voltage to be applied to the two electrodes. For example, vertical vibrations and bending vibrations are repeated alternately by the voltage applied thereto. The vertical vibrations change the length of the plate-shaped member 151 in an axial direction, and the bending vibrations change the shape of the plate-shaped member into a substantially S-shape. By repeating changes alternately, the rotor gear 128 is rotated in a predetermined direction.

Referring also to FIG. 4, the rotor gear 128 includes pinions configured to rotate integrally at different positions in terms of the height direction of the micro pump 1, and the pinions engage a tooth portion of an intermediate gear 127 to rotate the intermediate gear 127. The intermediate gear 127 also includes pinions configured to rotate integrally at different position in terms of the height direction of the micro pump, and the pinions engage the tooth portion rotating integrally with an output shaft 126. A supporting shaft of the rotor gear 128, and a supporting shaft of the intermediate gear 127 and an output shaft 126 are individually pivotally supported rotatably by a gear train receipt 125 fixed to the main body 10 (FIG. 5).

The cam 121 is held by the output shaft 126, which is pivotally supported by bearings 129, so as to be integrally rotatable (FIG. 4). The cam 121 is also allowed to rotate together with the rotation of the output shaft 126. Accordingly, a motive force from the piezoelectric motor 150 is transmitted to the cam 121.

As illustrated in FIG. 6, a hook catch 171 is provided at one end of the main body 10, and two hook insertion ports 172 are provided at the other end thereof. A fixed hook 271 of the cartridge 20 is hooked on the hook catch 171, and a fixed hook 272 is hooked on the hook insertion ports 172, so that the cartridge 20 is fixed to the main body 10 (FIG. 2 and FIG. 4).

At this time, since a packing 273 (FIG. 4) is fitted to a grove portion of an outer periphery of an upper surface of the cartridge base 210, if the main body 10 and the cartridge 20 are fixed, a space defined thereby is sealed and is prevented from entry of liquid or the like.

As illustrated in FIG. 6, the main body 10 includes a power source unit 180 on a back surface 110a thereof. The power source unit 180 includes a secondary battery storage 181 and a secondary battery 184 (FIG. 4). The secondary battery storage 181 includes a battery plus terminal 182 and a battery minus terminal 183, and a predetermined power supply is enabled to respective portions of the main body 10 by an insertion of the secondary battery 184 into the secondary battery storage.

Subsequently, the cartridge 20 will be described with reference to FIG. 7.

The cartridge 20 includes the cartridge base 210, a cartridge base holder 240, and respective portions provided on the cartridge base 210. The cartridge base 210 constitutes part of a storage portion 290 together with a reservoir film 250 as described later (FIG. 4).

The cartridge base 210 of the cartridge 20 includes a finger unit 220 on an upper surface thereof. The finger unit 220 includes a finger base 227, fingers 222, a tube 225, and a finger holder 226. An inlet connector 228 and a discharge connector 229 are provided on an upper surface of the cartridge base 210. The inlet connector 228 is a connector for intaking liquid into the finger unit 220, and the discharge connector 229 is a connector for discharging the liquid from the finger unit 220.

The finger base 227 is provided with a plurality of grooves, and the inlet connector 228 and the discharge connector 229 are inserted into the grooves. The finger base 227 is provided with a tube guide groove 227a formed thereon in an arcuate shape for guiding the tube 225 and storing the tube 225 (FIG. 10). The tube 225 is tightly connected to the inlet connector 228 and the discharge connector 229.

A plurality of finger guides 227b are formed inside the arc of the tube guide groove 227a. The fingers 222 are stored in the respective finger guides 227b. Accordingly, distal end portions 222a of the fingers 222 are disposed substantially perpendicularly with respect to the tube 225.

The finger holder 226 is fixed to an upper surface of the finger base 227 with a fixing screw, which is not illustrated. Accordingly, the fingers 222 are allowed to make a sliding movement only in the direction along the finger guides 227b.

In this manner, since the fingers 222 and the tube 225 are provided on the cartridge 20 side, even though the tube 225 having a different diameter is employed, the cartridge 20 combined with the fingers 222 having a length corresponding to the tube diameter may be provided. Accordingly, even though the cam 121 has a standardized size, a cam surface 121a of the cam 121 may be arranged suitably at positions abutting rear end portions 222b of the fingers 222.

A patch connecting needle 231 is provided on a side surface of the cartridge base 210 to allow liquid to be fed to the patch 30 via a patch septum 350 (FIG. 4). The patch connecting needle 231 communicates with the discharge connector 229 (FIG. 4). In contrast, the inlet connector 228 communicates with the storage portion 290, which will be described later, via a through hole provided in the cartridge base 210. Accordingly, the liquid of the storage portion 290 is allowed to pass through the inlet connector 228, the tube 225, and the discharge connector 229 and be supplied to the patch connecting needle 231.

A position of a distal end of the patch connecting needle 231 has the same height as the storage portion 290 in the height direction (FIG. 4). In this configuration, although the liquid passes through the tube 225 on the upper surface of the cartridge 20, the difference in height between the position of the distal end of the patch connecting needle 231 and the position of the storage portion 290 itself is small. Therefore, since the difference in positional energy may be reduced, the liquid stored in the storage portion 290 may be sent to the patch connecting needle 231 with small energy. This configuration is advantageous in the case where the piezoelectric motor 150 of an energy-saving type as described above is used.

As illustrated in FIG. 7 or FIG. 8, the cartridge 20 is provided with the reservoir film 250. The reservoir film 250 is interposed between the cartridge base 210 and a film holding unit 242 provided on a cartridge base holder 240 on a periphery thereof, and functions as a sealing member (packing). Accordingly, the storage portion 290 is provided between the reservoir film 250 and the cartridge base 210, whereby the liquid can be stored in the storage portion 290 without leaking therefrom.

It is also possible to fix the reservoir film 250 to the cartridge base 210 via welding, and fix the cartridge base holder 240 and the cartridge base 210 with each other.

The cartridge base 210 is formed of plastic, and the surface thereof on a side where the reservoir film 250 is provided has a curved shape. In this manner, although the storage portion 290 has a curved shape, since the film of the reservoir film 250 is deformable in accordance with the remaining amount of the liquid stored in the storage portion 290, the fluid can be squeezed out so as not to remain in the storing portion 290. At this time, the reservoir film 250 is preferably machined to have a curved shape extending along the curved shape described above. In this configuration, even though the amount of fluid in the storage portion 290 is reduced, since the reservoir film 250 is deformed corresponding to the curved surface, the liquid may be squeezed out without remaining therein.

The reservoir film 250 is formed of a multilayer film. At this time, an inner layer is preferably formed of polypropylene, and an outer layer is preferably selected from materials superior in gas barrier property. The reservoir film 250 is not limited thereto, and may be a film formed of, for example, a thermoplastic elastomer, or other materials adhered to the thermoplastic elastomer.

A cartridge septum 280 is provided on a lower surface of the cartridge 20 (FIG. 9). The cartridge septum 280 is inserted into a cartridge septum insertion hole 241 provided in the cartridge base holder 240 when the cartridge base 210 and the cartridge base holder 240 are assembled. One of the surfaces of the cartridge septum 280 is exposed to openings 340a and 360a of a patch base 340 and an adhesion tape 360 (FIG. 2 and FIG. 9), and the other surface communicates with a fluid inlet port 211. The fluid inlet port 211 is opened between the reservoir film 250 and the cartridge base 210. Therefore, the liquid injected via the cartridge septum 280 by using an infusion needle or the like is stored in the storage portion 290.

Subsequently, the patch 30 will be described with reference to FIG. 4 again. The patch 30 is provided with a catheter 310, an introduction needle 320, an introduction needle folder 321, an introduction needle septum 322, a port base 330, the patch base 340, the patch septum 350, and the adhesion tape 360.

The patch septum 350 is configured to supply the liquid into the patch 30 by inserting the patch connecting needle 231 thereto as will be described later. The patch septum 350 is provided on a side wall portion of the patch 30, and when the cartridge 20 is mounted toward the side surface of the patch 30, the patch connecting needle 231 penetrates through the patch septum 350.

A septum such as the patch septum 350 is formed of materials which closes a hole formed by the penetration of the needle or the like (for example, silicone rubber, isoprene rubber, butyl rubber, and the like). Accordingly, even though the needle is inserted in and pulled out from the septum, the liquid or the like is not leaked out from the septum.

The catheter 310 is a tube for infusing liquid. Part of the catheter 310 is held by the port base 330, and is partly exposed to a lower side of the port base 330. When infusing liquid by using the patch 30, the exposed portion of the catheter 310 is indwelled in the interior of the biological body or the like, and the liquid is continuously infused. Therefore, the catheter 310 is formed of a soft material such as fluorine resin, polyurethane resin superior in adaptation with the biological body.

The introduction needle 320 is a member having a hollow thin needle shape having an outer diameter smaller than an inner diameter of the catheter 310. The introduction needle 320 is inserted into the catheter 310 before use. A sharp side of the introduction needle 320 exposes downward of the catheter 310, and the other end side is fixed to the introduction needle folder 321. Before use, the introduction needle 320 is inserted into the introduction needle septum 322 fixed in the port base 330.

In this configuration, the introduction needle 320 is pulled out from the catheter 310 by the introduction needle folder 321 being pulled out from the port base 330. However, the liquid flowing from the patch connecting needle 231 is not leaked from the introduction needle septum 332 side, but passes through the catheter 310 and flows into the biological body.

The patch 30 is provided with the patch base 340. The patch base 340 is fixed to the port base 330, and is provided with a cartridge fixing member 341, and is capable of fixing the cartridge 20 to the patch 30. When the cartridge 20 is connected to the patch 30, the cartridge 20 is slid from the left side in FIG. 2 with respect to the patch 30. Then, the patch connecting needle 231 provided on the cartridge 20 penetrates through the patch septum 350 and is inserted into the patch 30.

The patch base 340 is provided with the adhesion tape 360 on the lower surface thereof, then, the micro pump 1 can be adhered to the biological body or the like.

In the case where the main body 10 and the cartridge 20 are assembled, the cam 121 of the main body 10 is inserted into a cam storage unit 227c of the finger bases 227. Accordingly, the cam surface 121a of the cam 121 is arranged at a position facing the rear end portions 222b of the fingers 222. Then, the cam surface 121a comes into abutment with the rear end portions 222b of the fingers 222 by the rotation of the cam 121, so that the fingers 222 may be brought into a sliding motion.

FIG. 10 is an explanatory drawing of a rotary finger pump. Four cam protrusions are formed on the cam 121. The cam protrusions each have a shape making up the transition from the lowest point gradually upward to the highest point of the cam protrusion, and from the highest point to the lowest point of an adjacent cam protrusion. In this shape, when the cam 121 rotates, the distal end portions 222a of a plurality of the fingers 222 presses the tube 225 in a direction from the inlet connector 228 side toward the discharge connector 229 side in sequence. Consequently, the liquid in the tube 225 is fed from the inlet connector 228 side to the discharge connector 229 side.

FIG. 10 illustrates an ultrasonic sensor 122 and a pressure detecting member 260, which will be described later. A cylindrical portion 2601a included in the pressure detecting member 260 is illustrated. As will be described later, the ultrasonic sensor 122 is arranged so that an ultrasonic wave sending and receiving surface faces a side wall of the cylindrical portion 2601a, and is configured to be capable of detecting displacement of the cylindrical portion 2601a.

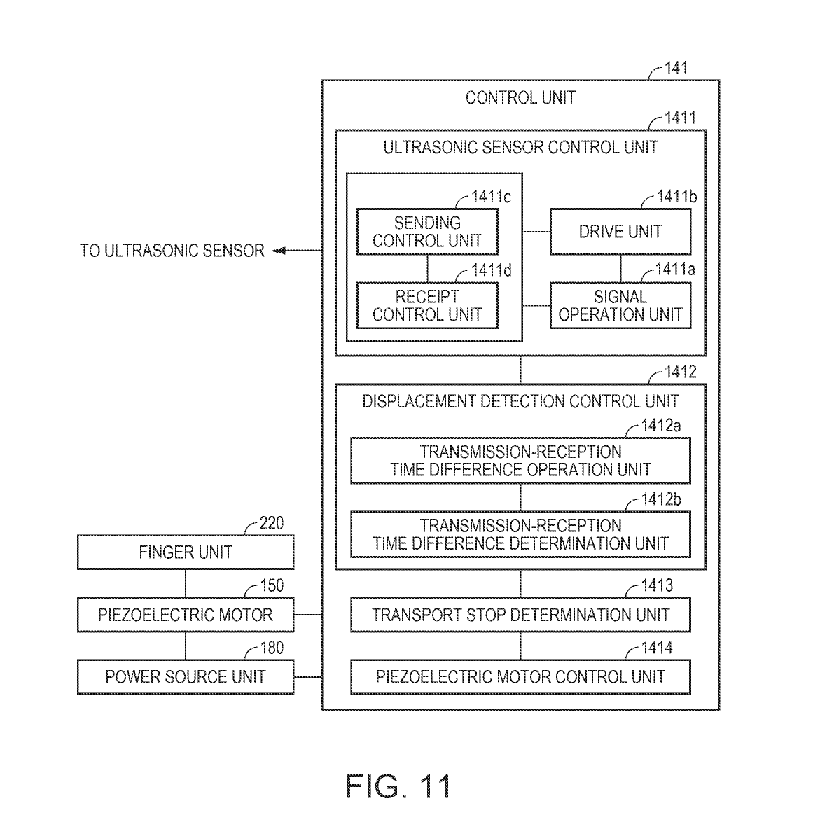

FIG. 11 is a block diagram of a control unit 141 in the micro pump 1 of the first embodiment. The control unit 141 is connected to the piezoelectric motor 150. The control unit controls the piezoelectric motor 150 physically connected to the finger unit 220, and controls the amount of transport volume of liquid in the micro pump 1. The control unit 141 is connected to the power source unit 180 and receives a supply of electric power.

The control unit 141 includes an ultrasonic sensor control unit 1411, a displacement detection control unit 1412, a transport stop determination unit 1413, and a piezoelectric motor control unit 1414.

The ultrasonic sensor control unit 1411 controls the ultrasonic sensor 122, which will be described later, causes the ultrasonic sensor 122 to send and receive ultrasonic waves, and obtains a propagation time. The ultrasonic sensor control unit 1411 includes a signal operation unit 1411a, a drive unit 1411b, a sending control unit 1411c, and a receipt control unit 1411d.

The signal operation unit 1411a generates a waveform such as a square wave used for the ultrasonic wave to be sent. The drive unit 1411b drives the sending control unit 1411c and the receipt control unit 1411d. The sending control unit 1411c controls the ultrasonic sensor 122 to send an ultrasonic wave composed of square waves to a wall surface of the cylindrical portion 2601a, which will be described later. The receipt control unit 1411d receives an ultrasonic wave reflected from the wall surface of the cylindrical portion 2601a.

The displacement detection control unit 1412 is a control unit configured to detect displacement of the cylindrical portion 2601a on the basis of a propagation time of the ultrasonic wave. The displacement detection control unit 1412 includes a transmission-reception time difference operation unit 1412a and a transmission-reception time difference determination unit 1412b.

The transmission-reception time difference operation unit 1412a computes a propagation time from the sending of the ultrasonic wave until the reception of a reflected wave. The transmission-reception time difference determination unit 1412b obtains an amount of change of the propagation time on the basis of a plurality of the obtained propagation times. As will be described later, when the cylindrical portion 2601a is displaced, the propagation time changes. In other words, obtaining the amount of change of the propagation time is equivalent to detection of the displacement of the cylindrical portion 2601a.

The transport stop determination unit 1413 determines a condition of transport of liquid on the basis of the amount of change of the propagation time. The transport stop determination unit 1413 determines whether or not the amount of change of the propagation time exceeds a predetermined threshold value. When the amount of change of the propagation time exceeds the predetermined threshold value, it is determined that the liquid is clogged, and hence the displacement exceeding the predetermined amount occurs in the cylindrical portion 2601a.

The piezoelectric motor control unit 1414 is a control unit configured to control the piezoelectric motor 150 in accordance with the result of determination of the transport stop determination unit 1413. The piezoelectric motor control unit 1414 causes the piezoelectric motor 150 to operate as normal when the amount of change of the propagation time does not exceed the predetermined threshold value. In contrast, when the amount of change of the propagation time exceeds the predetermined threshold value, the operation of the piezoelectric motor 150 is stopped.

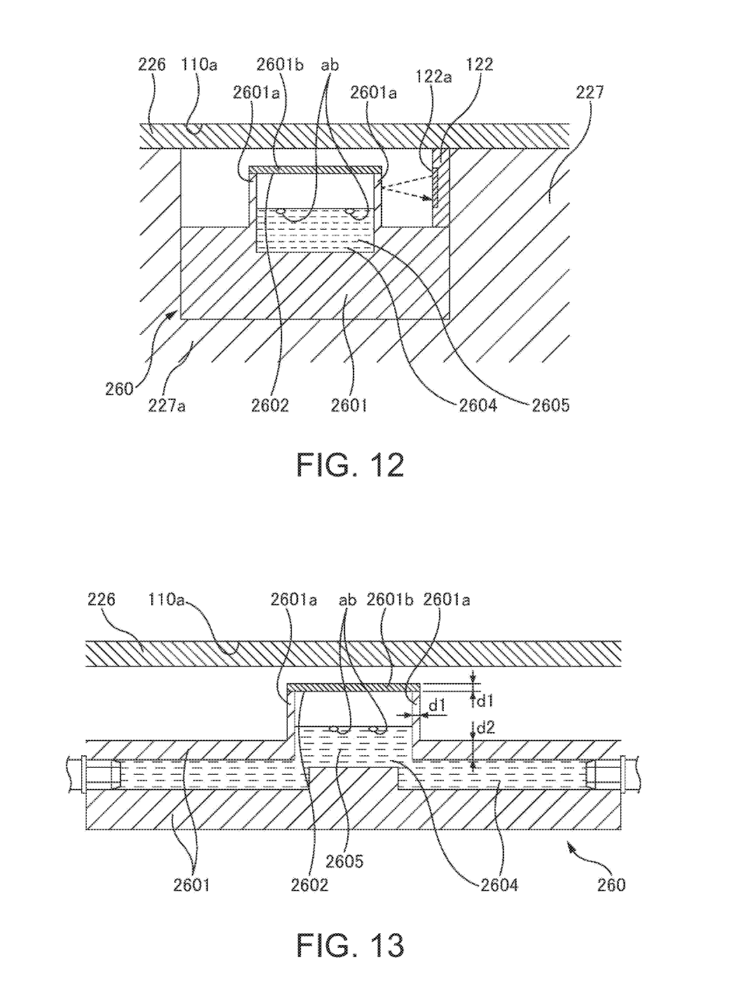

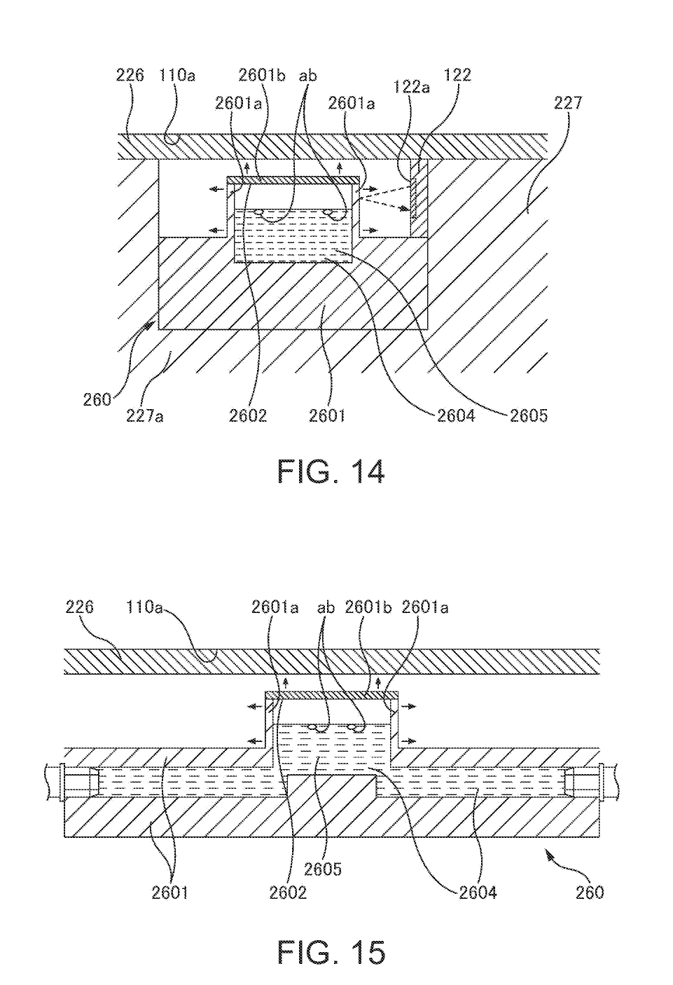

FIG. 12 is a first cross-sectional view taken along a B-B line in FIG. 3 (first embodiment). FIG. 13 is a first cross-sectional view taken along a C-C line in FIG. 3 (first embodiment). FIG. 14 is a second cross-sectional view taken along the B-B line in FIG. 3 (first embodiment). FIG. 15 is a second cross-sectional view taken along the C-C line in FIG. 3 (first embodiment). FIG. 12 and FIG. 13 illustrate states before the flow channel of the liquid is clogged. In contrast, FIG. 14 and FIG. 15 illustrate states when the flow channel of the liquid is clogged. Detection of clogging of the first embodiment will be described with reference to these drawings.

The drawings illustrate the ultrasonic sensor 122 and the pressure detecting member 260. The ultrasonic sensor 122 includes an ultrasonic module 122a configured to send and receive ultrasonic waves.

The pressure detecting member 260 includes a flow channel member 2601 and the cylindrical portion 2601a provided on the flow channel member 2601. The cylindrical portion 2601a is closed on top by a lid member 2601b included in the cylindrical portion 2601a. The flow channel member 2601 includes a communication hole 2604 penetrating in a direction of liquid flow, and a through hole 2605 penetrating through part of the communication hole 2604 from an upper part thereof.

The cylindrical portion 2601a is a cylindrical portion extending in the direction of the through hole 2605, whereby a space is generated in the through hole 2605. In this space, a gas layer and a liquid layer exist separately. Then, the liquid layer constitutes part of the communication hole 2604, and the liquid flows therethrough. The cylindrical portion 2601a may be formed into a cylindrical shape, but is not limited thereto.

As illustrated in FIG. 13, a thickness d1 of the side wall of the cylindrical portion 2601a is formed thinner than a thickness d2 of a thinned portion of the flow channel member 2601. Then, the rigidity of the cylindrical portion 2601a is set to be lower than the rigidity of the flow channel member 2601. Accordingly, as will be described later, when an internal pressure is increased, the cylindrical portion 2601a is liable to be deformed more than the flow channel member 2601. In order to set the rigidity of the cylindrical portion 2601a lower than the rigidity of the flow channel member 2601, the material of the cylindrical portion 2601a may be differentiated from the material of the flow channel member 2601. Rigidity of the cylindrical portion 2601a is preferably lower than rigidity of the tube 225.

The pressure detecting member 260 is fitted along the tube guide groove 227a in the finger base 227. The tube 225 is fixed to an upstream end and a downstream end of the communication hole 2604 of the pressure detecting member 260.

In contrast, the ultrasonic sensor 122 is fixed to a side wall of the tube guide groove 227a after the pressure detecting member 260 is fitted to the tube guide groove 227a in the finger base 227. At this time, an ultrasonic wave sending and receiving surface of the ultrasonic module 122a is fixed so as to face the wall surface of the cylindrical portion 2601a. The ultrasonic sensor 122 is connected to the control unit 141 via a connector or the like, which is not illustrated.

In the case where the liquid flow channel is clogged when a flow is occurring in the tube 225 by the finger unit 220, the internal pressure of the flow channel member 2601 is enhanced (FIG. 14, FIG. 15). At this time, since the rigidity of the cylindrical portion 2601a is lower than the rigidity of the flow channel member 2601, the cylindrical portion 2601a is deformed more significantly in the direction of expansion by the internal pressure. In other words, the distance between the cylindrical portion 2601a and the ultrasonic sensor 122 is reduced.

In the first embodiment, an ultrasonic wave including square waves is sent from the ultrasonic module 122a at every predetermined period (for example, every 5 minutes) by control of the ultrasonic sensor control unit 1411. The ultrasonic wave sent from the ultrasonic module 122a is reflected by the wall surface of the cylindrical portion 2601a, which is a measurement object, and the reflected wave is detected by the ultrasonic module 122a. The propagation time from the sending of the ultrasonic wave until the reception of the reflected wave is obtained by the displacement detection control unit 1412 (FIG. 11).

In this manner, the propagation time is obtained at every predetermined period. Then, the amount of change of the propagation time is obtained by the transmission-reception time difference operation unit 1412a on the basis of a plurality of the propagation times. For example, as the amount of change of the propagation time, how much the propagation time has changed with reference to the propagation time obtained at the beginning is obtained.

The transport stop determination unit 1413 determines that the tube 225 is clogged when the obtained amount of change of the propagation time exceeds a predetermined threshold value. Then, the piezoelectric motor control unit 1414 forcedly stops driving of the piezoelectric motor 150.

In this configuration, the condition of transport of the liquid may be determined by the micro pump 1. Then, the driving of the piezoelectric motor 150 may be stopped on the basis of the result of determination.

As described above, the rigidity of the cylindrical portion 2601a of the pressure detecting member 260 is lower than the rigidity of the flow channel member 2601 as described in the first embodiment. Therefore, if the clogging occurs downstream, and the internal pressure is increased, the cylindrical portion 2601a is deformed significantly. Therefore, the clogging is detected with higher sensitivity by detecting the displacement of the cylindrical portion 2601a.

In addition, the flow channel member 2601 includes the cylindrical portion 2601a, and includes an air layer in the cylindrical portion 2601a. Air in the air layer is readily compressed in comparison with the liquid, and hence even in the case where an abrupt change is generated in the condition of transport of the liquid, the abrupt change may be alleviated by the air layer. Accordingly, a damage generated from an excessive deformation of the cylindrical portion 2601a may be restrained.

Liquid flow through the communication hole 2604, and the liquid may contain air bubbles ab. There is a demand not to inject air bubbles ab into the biological body. In response to the demand, in the first embodiment, as described above, the cylindrical portion 2601a is provided and a gas layer is provided in the interior thereof. In this configuration, the air bubbles ab contained in the liquid may be caught in the interior of the cylindrical portion 2601a.

In the description given above, the side wall surface of the cylindrical portion 2601a is irradiated with ultrasonic wave. However, the lid member 2601b of the cylindrical portion 2601a may be irradiated with the ultrasonic wave. If the internal pressure is heightened, the entire cylindrical portion is expanded and the position of the lid member 2601b will be displaced.

### Second Embodiment

In the first embodiment described above, a displacement of the cylindrical portion 2601a is obtained by using the ultrasonic sensor 122. In a second embodiment, the displacement of the cylindrical portion 2601a is obtained by using a strain gauge 123. Different points from the first embodiment will be described below.

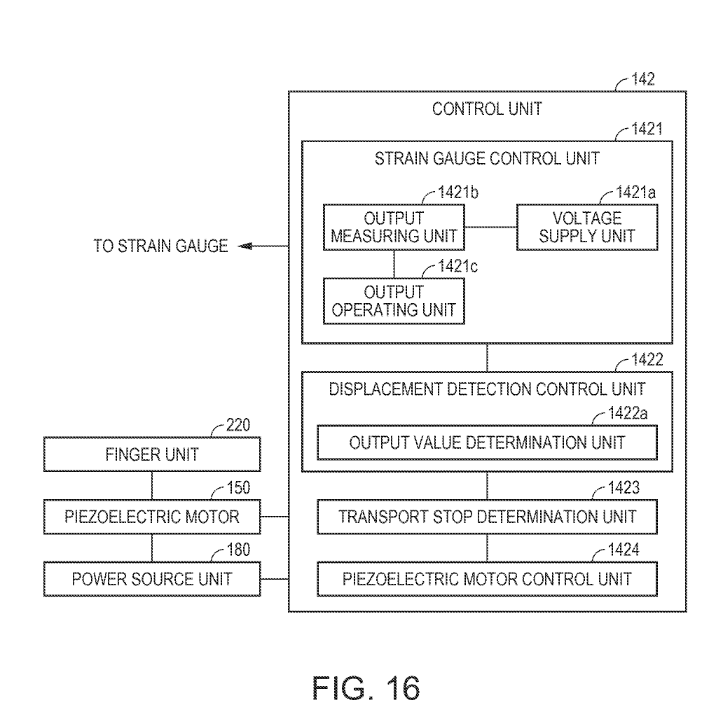

FIG. 16 is a block diagram of a control unit 142 in a micro pump 1 of the second embodiment. In the second embodiment, the control unit 142 illustrated in FIG. 16 is used instead of the control unit 141 used also in the first embodiment. In the control unit 142 of the second embodiment, a different point from the control unit 141 of the first embodiment is in that a strain gauge control unit 1421 and a displacement detection control unit 1422 are provided.

The strain gauge control unit 1421 includes a voltage supply unit 1421a, an output measuring unit 1421b, and an output operation unit 421c. The voltage supply unit 1421a applies voltage to the strain gauge 123, which will be described later. The output measuring unit 1421b measures a current value in the strain gauge 123. The output operation unit obtains a resistance value of the strain gauge 123 on the basis of the applied voltage value and the obtained current value.

The displacement detection control unit 1422 includes an output value determination unit 1422a. The output value determination unit 1422a obtains the amount of displacement of the cylindrical portion 2601a on the basis of the resistance value of the strain gauge 123.

A transport stop determination unit 1423 is a determining unit configured to determine the condition of transport of liquid on the basis of the amount of displacement of the cylindrical portion 2601a, and determining whether or not the transport of the liquid is stopped according to the result of determination. The transport stop determination unit determines whether or not the amount of displacement of the cylindrical portion 2601a exceeds a predetermined threshold value. When the amount of displacement of the cylindrical portion 2601a exceeds the predetermined threshold value, it is determined that the liquid is clogged, and hence the displacement exceeding the predetermined value occurs in the cylindrical portion 2601a.

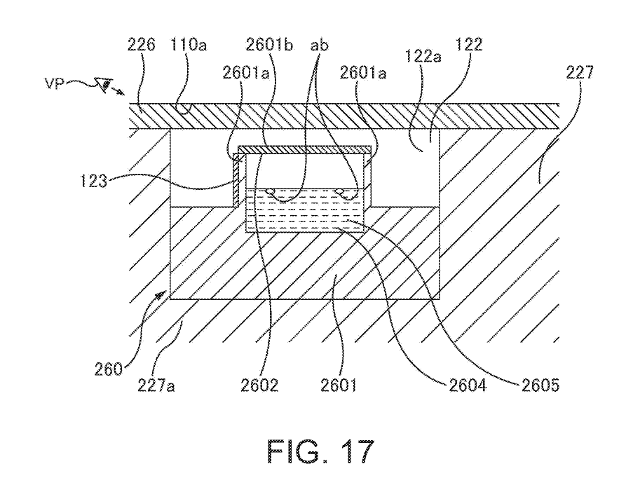

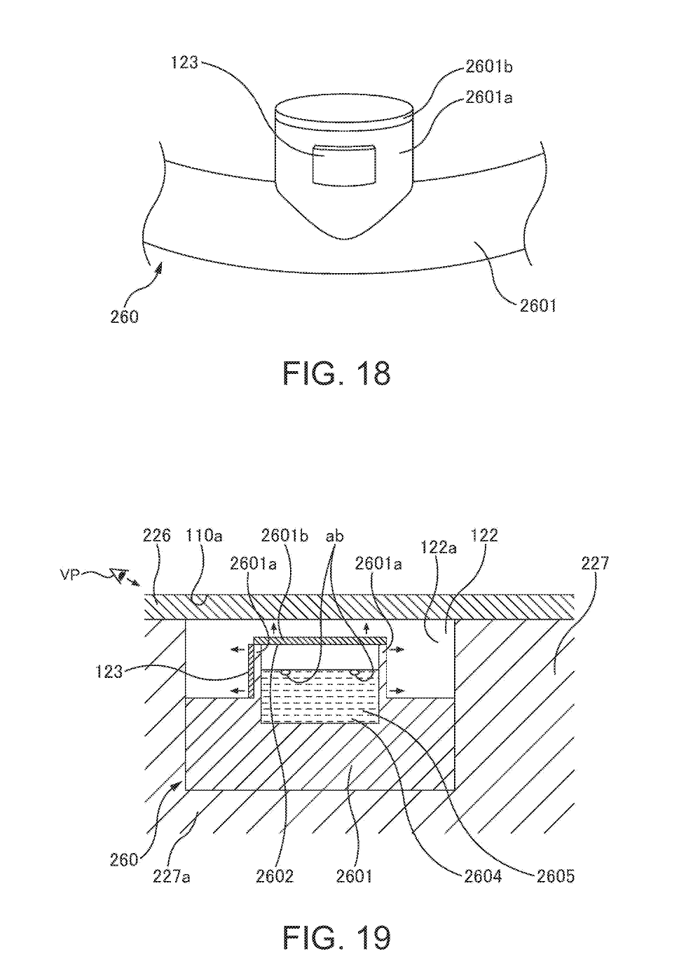

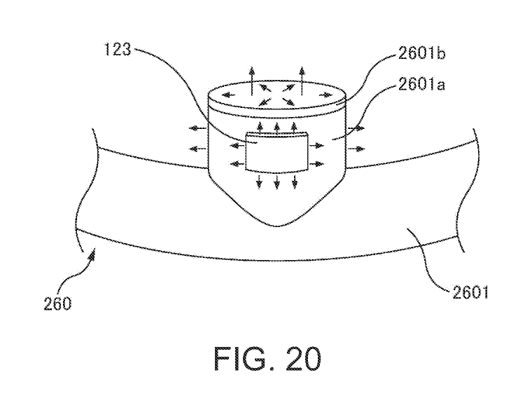

FIG. 17 is a first cross-sectional view taken along the B-B line in FIG. 3 (second embodiment). FIG. 18 is a first perspective view of the pressure detecting member 260 (second embodiment). The perspective view of FIG. 18 is a perspective view of the pressure detecting member 260 when viewing from a view point VP in FIG. 17 in a perspective manner. FIG. 19 is a second cross-sectional view taken along the B-B line in FIG. 3 (second embodiment). FIG. 20 is a second perspective view of the pressure detecting member 260 (second embodiment). The perspective view of FIG. 20 is a perspective view of the pressure detecting member 260 when viewing from a view point VP in FIG. 19 in a perspective manner.

FIG. 17 and FIG. 18 illustrate states before the flow channel of the liquid is clogged. In contrast, FIG. 19 and FIG. 20 illustrate states when the flow channel of liquid is clogged. Detection of clogging of the second embodiment will be described below with reference to these drawings.

In FIG. 17 to FIG. 20, a point different from the first embodiment is in that the ultrasonic sensor 122 is removed, and the strain gauge 123 is adhered to the side wall of the cylindrical portion 2601a instead. Other configurations of the flow channel member 2601 and the like are the same as that described in the first embodiment, and hence description will be omitted. The strain gauge 123 is connected to the control unit 142 via a connector or the like, which is not illustrated.

In the case where the liquid flow channel is clogged when a flow is occurring in the tube 225 by the finger unit 220, the internal pressure of the flow channel member 2601 is enhanced (FIG. 19, FIG. 20). At this time, since rigidity of the cylindrical portion 2601a is lower than rigidity of the flow channel member 2601, the cylindrical portion 2601a is deformed more significantly than the flow channel member 2601 due to the internal pressure thereof.

In the second embodiment, voltage is applied to the strain gauge 123 by the voltage supply unit 1421a at every predetermined period (for example, every 5 minutes) by control of the strain gauge control unit 1421, and resistance values thereof are obtained. The amount of displacement of the cylindrical portion 2601a is obtained by the displacement detection control unit 1422 on the basis of an obtained resistance value (FIG. 16).

The transport stop determination unit 1423 determines that the tube 225 is clogged when the obtained amount of displacement of the cylindrical portion 2601a exceeds a predetermined threshold value. Then, a piezoelectric motor control unit 1424 forcedly stops driving of the piezoelectric motor 150.

In this configuration, the displacement of the cylindrical portion 2601a may be measured directly to determine the condition of transport of liquid in the micro pump 1. Then, the driving of the piezoelectric motor 150 may be stopped on the basis of the result of determination.

In the description given above, the strain gauge 123 is mounted on the side wall surface of the cylindrical portion 2601a. However, the strain gauge 123 may be mounted on the lid member 2601b of the cylindrical portion 2601a. Because if the internal pressure is heightened, the entire cylindrical portion is expanded, and the lid member 2601b is also expanded and deformed.

### Other Examples

Since the micro pump 1 described above can achieve small sizes and thin profiles, and cause a very small amount of flow stably and continuously. Therefore, it is suitable for medical practices such as development of new medicines, or drug deliveries by mounting inside biological bodies or on the surfaces of the biological bodies. The micro pump 1 may be used in several mechanical apparatuses by mounting in the apparatus or in the exterior of the apparatus for transferring fluid such as water, saline solution, drug solution, oils, aromatic liquid, ink, gas, and the like. Furthermore, the micro pump itself may be used for a flow and a supply of fluid as a stand-alone unit.

The embodiment described above is for facilitating the understanding of the invention, and is not for interpreting the invention in a limited range. It is needless to say that the invention may be modified or improved without departing the scope of the invention and equivalents are included in the invention.

The entire disclosure of Japanese Patent Application No. 2013-207970, filed Oct. 3, 2013 is expressly incorporated by reference herein.

What is claimed is:

1. A fluid infusing apparatus comprising:
a flow channel member configured to transport a fluid;
a cylindrical portion provided on the flow channel member,
a measuring unit configured to measure a displacement of the cylindrical portion;
a determining unit configured to determine a change of a condition of transport of the fluid on the basis of the displacement of the cylindrical portion.

2. The fluid infusing apparatus according to claim 1, wherein
the measuring unit includes at least one of an ultrasonic sensor and a strain gauge.

3. The fluid infusing apparatus according to claim 1, wherein
the cylindrical portion includes a lid portion, and
an air layer is provided between the lid portion and the fluid.

4. The fluid infusing apparatus according to claim 1, wherein
rigidity of the cylindrical portion is lower than rigidity of the flow channel member.

5. The fluid infusing apparatus according to claim 1, wherein
a thickness of the cylindrical portion is lower than a thickness of the flow channel member.

6. The fluid infusing apparatus according to claim 1, wherein
rigidity of a tube to be connected to the flow channel member is higher than at least the rigidity of the cylindrical portion.

7. The fluid infusing apparatus according to claim 1, further comprising:
the cylindrical portion extends from the flow channel member in a direction orthogonal to a flow channel of the flow channel member.

8. The fluid infusing apparatus according to claim 1, further comprising:
a pump for causing the fluid to flow, wherein
the determining unit controls an operation of the pump on the basis of the determined condition of transport of the fluid.

9. A fluid infusing apparatus comprising:
a flow channel member provided with a through hole as a flow channel configured to allow the fluid to flow therein and configured to transport the fluid,
the through hole including:
a first area extending in the flowing direction and covered with a first wall;
a second area extending in a direction intersecting a direction of extension of a first hole, being connected to the first area, and being covered with a second wall;
a measuring unit configured to measure a displacement of the second wall; and
a determining unit configured to determine a condition of transport of the fluid on the basis of the displacement of the second wall measured by the measuring unit.

10. The fluid infusing apparatus according to claim 9, wherein
the second wall includes a portion protruding from the first wall which covers the first area; and
the measuring unit is configured to measure a displacement of the portion protruding included in the second wall.

11. The fluid infusing apparatus according to claim 9, wherein
an air layer is provided between the second wall and the fluid in the second area of the through hole.

12. The fluid infusing apparatus according to claim 9, wherein
rigidity of the second wall is lower than rigidity of the first wall.

13. The fluid infusing apparatus according to claim 9, wherein
a thickness of the second wall is lower than a thickness of the first wall.

14. The fluid infusing apparatus according to claim 9, wherein
the rigidity of the second wall is lower than the rigidity of the tube to be connected to the flow channel member.

15. The fluid infusing apparatus according to claim 9, wherein
a pump for causing the fluid to flow, wherein
the determining unit controls an operation of the pump on the basis of the determined condition of transport of the fluid.

16. A transporting state determination method for a fluid in a fluid infusing apparatus including a flow channel member configured to transfer the fluid, and a cylindrical portion provided on the flow channel member, comprising:
measuring a displacement of the cylindrical portion; and
determining a condition of transport of the fluid on the basis of the measured displacement of the cylindrical portion.
